(19) United States
(12) Patent Application Publication
(10) Pub. No.: US 2004/0087021 A1
(43) Pub. Date: May 6, 2004

(54) Compositon for culturing cells, in particular animal or tissue cells, and a culture medium comprisiing such a composition

(76) Inventor: Antoine Heron, Halluin (FR)

Correspondence Address: BAKER BOTTS, LLP, 910 LOUISIANA, HOUSTON, TX, 77002-4995, US

(21) Appl. No.: 10/685,609

(22) Filed: Oct. 15, 2003

(30) Foreign Application Priority Data
Oct. 16, 2002 (FR) FR 02/12878

(51) Int. Cl. C12N 5/02

(52) U.S. Cl. 435/404

(57) ABSTRACT

The invention concerns a composition for the culture of cells, in particular animal cells or tissues, free from animal proteins other than recombinant proteins, of the type including an albumin substitute, a transferrin substitute and an insulin substitute, the said composition being characterized in that the albumin substitute is polyethylene glycol in quantities greater than or equal to 1% by weight. The invention also includes reduced albumin compositions in which part of the albumin is replaced by polyethylene glycol.

The invention also includes a culture medium including such a composition and methods of culturing cells or tissues in such compositions or media.

### PRIORITY

This application claims foreign priority of the French application FR 0212878 filed on Oct. 16, 2002.

### FIELD OF THE INVENTION

The invention concerns a composition for culturing cells, in particular animal or tissue cells, and a culture medium comprising such a composition. In particular, the invention concerns a composition and a cell culture medium containing no animal proteins other than recombinant ones.

### BACKGROUND OF THE INVENTION

In the field of in vitro culture media for cells which can be directly used in humans, media are already known whose main constituent is serum. These media do however have drawbacks. First of all, the composition of such media is only partially known, which poses problems in particular in terms of functional reproducibility between different serums and between different batches of the same serum.

In addition, serums are a potential vector of pathogenic agents. In particular, when the serum is of bovine origin, the risk of contamination by prions always exists.

To remedy these drawbacks, culture media without serum, or “serum-free media”, have been proposed. The media have as their essential constituents a purified natural protein serving a serum substitute, such as albumin, transferrin and insulin. However, it is still difficult to achieve standardization of such media, and the risks of contamination by pathogenic agents are not insignificant. In addition, the presence of proteins in these mediums creates difficulties during sterilizing filtration steps, in particular during filtration on membranes with a porosity of 0.2 microns or below.

Moreover, the culture media containing such proteins have a high production cost.

In order to attempt to resolve these and other drawbacks, a culture medium comprising an albumin substitute, a transferrin substitute and an insulin substitute is known, in particular from the document WO-98/30679. However, such media are not free from proteins, in that in particular the albumin substitutes used are themselves proteins.

This type of medium, although not containing any serum, does not therefore completely dispense with the drawbacks related to the use of proteins, such as the risk of contamination by pathogenic agents and the difficulties in standardization.

### SUMMARY OF THE INVENTION

The invention includes a composition and a culture medium containing as an albumin substitute, polyethylene glycol in a given concentration range, so as to fulfil desirable functions.

In one embodiment, the invention relates to a composition for the culture of cells or tissues using an albumin substitute. The albumin substitute includes polyethylene glycol at a concentration of at least approximately 1% by weight.

In more specific embodiments, the composition may also include transferrin substitute and an insulin substitute, and may be free from animal proteins other than recombinant proteins. The composition may also contain sodium erythorbate.

In another embodiment, the invention relates to a composition containing a protein and serum free cell culture base and at least 1% polyethylene glycol by weight. In more specific embodiments, the composition is free from animal proteins other than recombinant proteins. The polyethylene glycol may substitute for all or part of the amount of albumin normally required to culture cells or tissues in the composition. In some embodiments, the composition contains substantially no albumin.

The composition may also contain a transferrin substitute and an insulin substitute. Sodium erythorbate may be added in amount of less than 0.1% by weight, more specifically less than 5*10⁻³% by weight.

Polyethylene glycol may be present in an amount of between 1% and 5% by weight, more specifically between 1% and 3% by weight. The polyethylene glycol may have a molecular weight of between 50 and 100,000 daltons, more specifically it may have amolecular weight of approximately 20,000 daltons. In certain embodiments, an additional substance may be complexed with the polyethylene glycol. This additional substance may a liquid or it may be a growth factor, inter alia.

In some specific embodiments, the composition may be in the form of a power, in other embodiments it may be a liquid.

In yet another embodiment, the invention relates to a cell or tissue culture medium including a protein and serum free cell culture base and at least 10 g/l polyethylene glycol.

In more specific embodiments, the medium is free from animal proteins other than recombinant proteins. The polyethylene glycol may substitutes for all or part of the amount of albumin normally required to culture cells or tissues in the medium. The medium may contain substantially no albumin. In other specific embodiments, it may contain albumin. The albumin concentration may be less than 5 g/l.

The medium may contain a transferrin substitute and an insulin substitute. It may also contain sodium erythorbate in an amount of less than 1 g/l, more specifically in an amount of .less than 50 mg/l.

The medium may contain polyethylene glycol in an amount of between 10 g/l and 50 g/l, more particularly between 10 g/l and 30 g/l. The polyethylene glycol may have a molecular weight of between 50 and 100,000 daltons, more particularly approximately 20,000 daltons. An additional substance may be complexed with the polyethylene glycol. This additional substance may be a liquid or a growth factor, inter alia.

Another embodiment of the invention relates to a method of culturing animal cells or tissues by placing the cells or tissues in a medium or composition described above.

For a more complete understanding of the present invention and the advantages thereof, reference is now made to the following description.

### DETAILED DESCRIPTION

The composition and medium of the present invention have the advantage, because they contain no protein of human, animal or non-recombinant origin, of having a well controlled and reproducible formulation and presenting little to no risk of transmission of pathogenic agents. In addition, the culture medium presents no difficulty in handling during the subsequent filtration steps.

In addition, the cost of such a composition and such a medium is often appreciably less than that of a composition or medium containing purified natural proteins.

One embodiment of the invention includes a composition for a culture of cells, in particular animal or tissue cells, free from animal proteins other than recombinant ones. Recombinant proteins may include an albumin substitute, a transferrin substitute and an insulin substitute, in which the albumin substitute is polyethylene glycol in a quantity greater than or equal to 1% by weight.

The composition may also include sodium erythorbate.

In another embodiment, the invention includes a culture medium containing such a composition.

Compositions according to the invention may be substantially free from animal proteins other than recombinant ones. All the natural proteins whose role is essential for cell culture, namely albumin, transferrin and insulin, may be replaced by non-natural components with equivalent properties. This reduces or eliminates problems with reproducibility or possible contamination by pathogenic agents.

Transferrin may be replaced in a known manner by an iron chelator such as iron sulphate, EDTA, EGTA or gluconic acid, so as to avoid the pasteurization phase made necessary when purified human transferrin is used.

The insulin may for example be replaced in a known manner by recombinant human insulin, or by a zinc salt.

Albumin in medium, and in particular injectable human albumin, causes, in addition to the drawbacks of reproducibility and cost mentioned above, problems related to the presence of stabilizing agents essential during viral inactivation steps carried out by heating.

In addition, injectable human albumin is not available in powder form, which explains why the media comprising such albumin are themselves available only in liquid form. Thus, when high volumes of these media are necessary, for example for biotechnological industrial usage, being available only in liquid form poses significant problems of production and logistics.

The use of polyethylene glycol as an albumin substitute has made it possible to dispense with these and other drawbacks because it is available both in powder form and in liquid form.

Polyethylene glycol possesses essentially the same functional properties as albumin in a cell culture. Within a culture medium, polyethylene glycol exerts in particular an osmotic effect, a stabilizing effect on the cell membranes and an effect on the maintenance of the cell viability during culture. In addition, it fulfils the role of detoxifier and trapper of free radicals, so as to prevent where necessary the peroxidation of the membrane lipids.

In order to fulfil the role of the albumin optimally, the quantity of polyethylene glycol introduced into the composition is, according to certain embodiments of the invention, greater than or equal to 1% by weight.

In particular embodiments, this quantity is between 1 and 5% by weight, and in particular between 1 and 3% by weight.

The polyethylene glycol used may have for example a molecular weight of between 50 and 100,000 daltons, in particular 20,000 daltons. The use of a polyethylene glycol of this type has the advantage of fully meeting the requirements of quality and safety necessary for therapeutic use. Polyethylene glycol polymers are normally used as solvents, synthesis intermediaries or excipients for cosmetic and pharmaceutical preparations and are therefore available in pharmaceutical grades.

According to one embodiment, the composition according to the invention includes sodium erythorbate, in a quantity less than or equal to 0.1% by weight, in particular between 10⁻⁶% and 0.1% by weight. More particularly, the quantity of sodium erythorbate is less than 5*10⁻³% by weight, and is in particular between 10⁻⁴% and 5*10⁻³% by weight. The introduction of such a molecule into the composition is intended to fulfil the role of a substitute for the antioxidant molecules. Sodium erythorbate has a high antioxidant capacity and is availale in pharmaceutical and food grades.

Moreover, the combination of polyethylene glycol and sodium erythorbate is particularly provide complementary protective benefits to cells and tissues in culture in vitro. These complementary benefits stem from the fact that sodium erythorbate has an antioxidant action on the biological medium constituting the environment of the cells and tissues in culture, while polyethylene glycol maintains the structural and functional integrity of the tissues and cells.

According to another embodiment, the composition according to the invention may also include a substance complexed with polyethylene glycol, by the so-called “pegylation” method. In many fields, polyethylene glycol is used as a carrier for molecules having a therapeutic function. The polyethylene glycol used may be of low molecular weight (less than 1000 g/mol) or high molecular weight (up to 100,000 g/mol).

The complexed substance may be a lipid, with the polyethylene as a lipid carrier. For example, polyethylene glycol with a molecular weight of 900 daltons can be fixed to cholesterol.

In addition, polyethylene glycol has the advantage of being able to be complexed with many other substances, its complexing possibilities are more extended than those of albumin. For example, polyethylene glycol with a molecular weight of 4500 or 10,000 daltons may be fixed to the human granulocyte CSF, which is a specific growth factor for haematopoietic cells, so as to increase the activity of the latter within a cell culture medium. Growth factors in culture media have the drawback of disappearing too rapidly. However, because of their complexing with polyethylene glycol, the stability of the growth factors may be improved.

Another embodiment of the invention is a culture medium including such a composition as described above.

This medium may be obtained by dissolving a composition like the one described above with a polyethylene glycol concentration greater than or equal to 10 g/l.

According to a specific embodiment, the polyethylene glycol concentration is between 10 and 50 g/l, and in particular between 10 and 30 g/l.

In addition, a culture medium may also be obtained by dissolving a composition including sodium erythorbate, the sodium erythorbate concentration being less than or equal to 1 g/l, and in particular between 0.01 mg/l and 1 g/l. More particularly, the sodium erythorbate concentration is less than 50 mg/l and is in particular between 1 and 50 mg/l.

Such culture media may be obtained by dissolving, in a given volume of liquid, a previously formulated composition, that is to say by the simultaneous dissolving of all the constituents of a composition according to the invention.

In a variant, the culture media can be obtained by the separate dissolution of certain constituents of a composition according to the invention, so as to obtain a culture medium in which the concentration of each constituent corresponds to the required value.

The general structure of one culture medium of the invention includes: a referenced basic formula, in particular IMDM (Iscove's Modified Dulbecco's Medium), DMEM (Dulbecco's Modified Eagle Medium), RPMI 1640 or others, (These bases are composed essentially of inorganic salts, amino acids, vitamins and other components, in particular glucose for its provision of energy and HEPES for its buffer capability.) basic supplements such as in particular non-essential amino acids, minerals and trace elements, recombinant human insulin or a substitute consisting of a zinc salt, an iron chelator as a transferrin substitute, polyethylene glycol (PEG), used at a concentration of less than 50 g/l of culture medium and in particular 10, 20, 25 and 30 g/l, possibly sodium erythorbate, at a concentration of between 0.01 mg/l and 1 g/l of culture medium and more precisely at concentrations of between 1 and 50 mg/l, molecular supplements specific to the growth and metabolic activities for each cell type cultivated.

Additionally, the medium does not exclude the use of molecules of vegetable origin. Insoluble lipids can also be added, in a free or complexed form, in particular with cyclodextrins. It is therefore possible to carry out cultures of animal cells or tissues using a medium according to the invention. By way of illustration, four examples of cell culture mediums are now described which are optimized for two distinct applications. Examples 1 and 2 describe a medium optimized for the expansion of stem cells and haematopoietic progenitors; Examples 3 and 4 define a medium optimized for the culture of hybridomas producing monoclonal.

The following examples are provided only to illustrate certain aspects of the invention and are not intended to embody the total scope of the invention or any aspect thereof. Variations of the exemplary embodiments of the invention below will be apparent to one skilled in the art and are intended to be included within the scope of the invention.

### EXAMPLES

### Example 1

### A Stem Cell and Haematopoietic Progenitor Expansion Medium

At the present time, the commercial media normally used for the culture of stem cells and haematopoietic progenitors belong to the range X-VIVO. According to the descriptions of the manufacturer itself, these media are composed of a basic formula, human albumin of pharmaceutical grade, recombinant human insulin and pasteurized human transferrin. The culture medium was developed in comparison with this manufacturing standard for culture media without serum.

A stem cell and haematopoietic progenitor expansion medium was defined according to the following formula:

an IMDM (Iscove's Modified Dulbecco's Medium) base composed of: anhydrous CaCl2 (165 mg/l), KCl (330 mg/l), KNO3 (0.076 mg/l), anhydrous MgSO4 (97.67 mg/l), NaCl (4505 mg/l), NaHCO3 (3024 mg/l), NaH2PO4.H2O (125 mg/l), Na2SeO3.5H2O (0.01 mg/l), glucose (4500 mg/l), HEPES (5958 mg/l), phenol red.Na (15 mg/l), sodium pyruvate (110 mg/l), L-Alanine (25 mg/l), L-Arginine.HCl (84 mg/l), L-Asparagine.H2O (28.40 mg/l), L-Aspartic acid (30 mg/l), L-Cystine.2HCl (91.24 mg/l), L-Glutamic acid (75 mg/l), L-Glutamine (584 mg/l), glycine (30 mg/l), L-Histidine.HCl.H2O (42 mg/l), L-Isoleucine (105 mg/l), L-Leucine (105 mg/l), L-Lysine.HCl (146 mg/l), L-Methionine (30 mg/l), L-Phenylalanine (66 mg/l), L-Proline (40 mg/l), L-Serine (42 mg/l), L-Threonine (95 mg/l), L-Tryptophan (16 mg/l), L-Tyrosine.2Na.2H2O (104.20 mg/l), L-Valine (94 mg/l), D-Biotin (0.013 mg/l), D-Ca Pantothenate (4 mg/l), choline chloride (4 mg/l), folic acid (4 mg/l), i-Inositol (7.2 mg/l), nicotinamide (4 mg/l), pyridoxine.HCl (4 mg/l), riboflavin (0.4 mg/l), thiamin.HCl (4 mg/l), vitamin B12 (0.013 mg/l),

basic supplements, in particular trace elements,

recombinant human insulin (1-100 mg/l) or an insulin substitute such as zinc chloride (0.1-10 mg/l),

iron gluconate (II) (50-1000 mg/l),

PEG 20,000 daltons (10-30 g/l),

specific supplements for the expansion of these cells such as: reduced glutathion (0.01-0.1 mg/l) and nucleosides (0.1-10 mg/l).

### Example 2

### Use of a Stem Cell and Haematopoietic Progenitor Expansion Medium

A stem cell and haematopoietic progenitor expansion medium which is identical to that defined in Example 1, also including sodium erythorbate (1-50 mg/l). Primitive haematopoietic cells (CD34+) extracted from umbilical cord blood were seeded at a concentration of 8000 cells per ml in a culture medium. Two combinations of growth factors (cytokines) were added in order to orient the growth of these cells:

cytokine cocktail N° 1 is based on SCF (50 ng/ml), TPO (50 ng/ml), FL (50 ng/ml), G-CSF (40 U/ml), GM-CSF (5 U/ml), IL-3 (1.7 U/ml) and IL-6 (10 U/ml),

cytokine cocktail N° 2 is based on SCF (50 ng/ml), TPO (50 ng/ml), FL (50 ng/ml), IL-3 (1.7 U/ml) and IL-6 (10 U/ml).

The cultures are incubated for 7 days at 37° C. in an atmosphere saturated with moisture comprising 95% air/5% CO₂. After 7 days of expansion, cell numbers and viability were determined and the cells seeded in a semi-solid medium (H4434, StemCell Technologies) in order to determine the rate of expansion of the clonogenic progenitors. The results are expressed in Table 1.

TABLE 1

| Medium | Cytokines | Cells at Day 0 | Cells at Day 7 | Viability | Expansion |
|---|---|---|---|---|---|
| IMDM base | Cocktail N° | 8000 | 505,000 | 88.7% | 63 × |
| | 1 | | | | |
| ME2* without | Cocktail N° | 8000 | 1,185,000 | 85.3% | 148 × |
| PEG 3% | 1 | | | | |
| ME2 with | Cocktail N° | 8000 | 1,755,000 | 93.8% | 219 × |
| PEG | 1 | | | | |
| 3% | | | | | |
| X-VIVO 15 | Cocktail N° | 8000 | 18,000,000 | 91.8% | 225 × |
| | 1 | | | | |
| ME2 with | Cocktail N° | 8000 | 907,000 | 97.5% | 113 × |
| PEG | 2 | | | | |
| 3% | | | | | |
| X-VIVO 15 | Cocktail N° | 8000 | 1,063,000 | 93.7% | 132 × |
| | 2 | | | | |

*ME2 = Medium according to Example 2

These results show that cytokine cocktail N° 1 causes a limited expansion of the cells (63 x) in the IMDM base used for preparing the medium according to the invention. The addition of the supplements with the exception of PEG proves the expansion results (148 x). Finally, the addition of PEG reinforces the competences of the medium according to the invention and makes it possible to obtain a degree of expansion (219 x) comparable with the reference medium (225 x).

This similarity in performance is confirmed with the use of cytokine cocktail N° 2. In all cases, the cell viability measured after 7 days of culture is excellent (85% to 97%). The clonogenic cultures have then demonstrated the quality of the culture medium according to Example 2 in terms of amplification of the clonogenic progenitors (progenitors capable of giving a colony of cells in vitro in a semi-solid medium).

An analysis of the colonies obtained is detailed in Table 2.

TABLE 2

| Medium | ME2 with PEG 3% | X-VIVO 15 |
|---|---|---|
| N° of colonies | (cytokine cocktail | (cytokine cocktail |
| for 400 cells | N° 2) | N° 2) |
| CFC total | 130/127* | 127/121 |
| CFU-Mixed | 7/4 | 1/1 |
| BFU-E | 79/73 | 83/80 |
| CFU-GM | 14/21 | 9/16 |
| CFU-G | 0/0 | 0/0 |
| CFU-M | 30/29 | 34/24 |

*Results obtained in duplicate (2 cultures analyzed per condition)

The clonogenicity index (the total number of colonies counted for 100 cells seeded) is invariant between the medium according to the invention and its reference (around 130 colonies counted for 400 cells tested). The distribution between the various types of colony is also similar. However an advantage is observed for the medium according to the invention: the progenitors of interest CFU-Mixed and CFU-GM are better represented. The impact of the PEG on the degree of expansion of the human cells CD34+ of cord blood and on the particular preservation of the clonogenic progenitors of the CFU-Mixed type was analyzed in other formulations of the medium according to the invention as well as in commercial reference media (results not shown).

### Example 3

### Medium for the Industrial Culture of Hybridomas

A medium for the industrial culture of hybridomas producing monoclonal antibodies was defined according to the following formulation:

an RPMI 1640 base composed of: Ca(NO3)2.4H2O (100 mg/l), KC1(400 mg/l), MgSO4.7H2O (100 mg/l), NaCl (5000 mg/l), NaHCO3 (2000 mg/l), NaH2PO4.7H2O (1512 mg/l), glucose (2000 mg/l), glutathione (reduced) (1 mg/l), HEPES (5957.4 mg/l), phenol red.Na (5 mg/l), L-Arginine (200 mg/l), L-Asparagine.H2O (50 mg/l), L-Aspartic acid (20 mg/l), L-Cystine (50 mg/l), L-Glutamic acid (20 mg/l), L-Glutamine (300 mg/l), glycine (10 mg/l), L-Histidine (15 mg/l), Hydroxy-L Proline (20 mg/l), L-Isoleucine (50 mg/l), L-Leucine (50 mg/l), L-Lysine.HCl (40 mg/l), L-Methionine (15 mg/l), L-Phenylalanine (15 mg/l), L-Proline (20 mg/l), L-Serine (30 mg/l), L-Threonine (20 mg/l), L-Tryptophan (5 mg/l), L-Tyrosine (20 mg/l) L-Valine (20 mg/l) p-Aminobenzoic acid (1 mg/l), D-Biotin (0.2 mg/l), D-Ca Pantothenate (0.25 mg/l), choline chloride (3 mg/l), folic acid (1 mg/l), i-Inositol (35 mg/l), nicotinamide (1 mg/l), pyridoxine.HCl (1 mg/l), riboflavin (0.2 mg/l), thiamin.HCl (1 mg/l), vitamin B12 (0.005 mg/l),

basic complements in particular for the trace elements,

an insulin substitute such as zinc chloride (0.01-10 mg/l),

iron gluconate (II) (50-1000 mg/l),

20,000 dalton PEG (10-30 g/l),

specific complements for the expansion of the hybridomas such as: nucleosides (0.1-10 mg/l), sodium pyruvate (1-100 mg/l), putrescine.2HCl (0.05-S mg/l), hypoxanthine (0. 1-10 mg/l).

### Example 4

### Use of Medium for Industrial Culture of Hybrodomas

A medium for the industrial culture of hybridomas producing monoclonal antibodies was defined in an identical fashion to that of Example 3, except with the addition sodium erythorbate (1-50 mg/l).

For the culture of hybridomas producing monoclonal antibodies, the IMDIA and DMEM/HAM-F12 (1/1) bases are also recommended. The complements of the medium according to the invention are then defined according to the base used: for example the base DMEM/HAM-F12 directly includes molecules such as putrescine, hypoxanthine, thymidine or sodium pyruvate.

The culture medium obtained from a composition of the present invention can therefore be used for the culture of animal cells and tissues with a therapeutic purpose. In particular the medium may be used for:

the culture of somatic stem cells, in particular stem cells and haematopoietic progenitors issuing from bone marrow, peripheral blood or umbilical cord blood,

the culture and activation of immune cells, in particular lymphocytes such as PBL (peripheral blood lymphocytes), LAK (lymphokine activated killer cells) and TIL (tumour infiltrating lymphocytes),

the culture of monocytes and macrophages,

the production of cells presenting antigens, in particular dendritic cells,

the culture of hepatocytes, in particular for the transplantation or preparation of a bioartificial liver,

the culture of islets of Langerhans, in particular in the context of the treatment of diabetes,

the expansion and differentiation of mesenchymatous stem cells, in particular in the context of the repair of bone deficits,

the culture of muscular cells, in particular in the context of the treatment of cardiac deficiencies,

the culture of neuron, somatic, foetal or embryo cells, and in particular in the context of the treatment of neurodegenerative illnesses,

the culture of corneas or other tissues,

the culture of embryonic stem cells,

the thawing, washing and freezing of cells and tissues for therapeutic purposes.

### Example 5

### Culture of Human Cells using Media of the Invention

According to the invention, for example for certain preparations of human cells for therapeutic purposes, it is also possible to use a culture medium in which PEG is partially substituted for albumin. To this end, the culture medium is obtained firstly by dissolving the composition according to the invention and secondly by adding the required quantity of albumin. For example, the quantity of albumin added to the culture medium may be less than 5 g/l, that is to say typically up to 50% of the albumin necessary is replaced with PEG.

To resolve the problems of potential contaminations related to the use of animal proteins, it is preferable then to use injectable human albumin (HSA).

For example, albumin plays a preponderant role in the expansion of haematopoietic stem cells (coming from normal peripheral blood (cells which are difficult to amplify), and it may also be useful for the recovery of dendritic cells (contribution to the removal of the cells from their plastic support).

An example of the production of a culture medium containing albumin is given below.

Primitive haematopoietic cells (CD34+) extracted from umbilical cord blood were seeded at a concentration of 8000 cells per ml in a culture medium. Growth factors (cytokines) were added in order to orient the growth of these cells: SCF (40 ng/ml), TPO (40 ng/ml), FL (40 ng/ml), IL-3 (1.7 U/ml) and IL-6 (10 U/ml). The cultures were incubated for 7 days at 37° C. in an atmosphere saturated with moisture at 95% air/5% CO2. After 7 days of expansion, the numbers and cell viability were determined. The results are detailed in Table 3.

TABLE 3

| | Cells at | Cells at | | |
|---|---|---|---|---|
| Medium | Day 0 | Day 7 | Viability | Expansion |
| Composition without | 8000 | 2,436,840 | 93.3% | 304 × |
| PEG + 1% HAS | | | | |
| Composition without | 8000 | 1,979,600 | 89.4% | 247 × |
| PEG + 0.3% HAS | | | | |
| Composition without | 8000 | 1,940,400 | 88.4% | 242 × |
| PEG + 0.2% HAS | | | | |
| Composition with | 8000 | 2,391,200 | 93.1% | 299 × |
| PEG 1.5% + 0.3% HAS | | | | |
| Composition with | 8000 | 2,152,840 | 92.2% | 269 × |
| PEG 1.5% + 0.2% HAS | | | | |

These results show that the degree of expansion of these cells decreases when the albumin content decreases (from 304 x to 242 x for an albumin content ranging from 10 g/l to 2 g/l). The partial substitution for the albumin, in particular by 20,000 daltons PEG, makes it possible to maintain the performance of the culture medium (for example: 299 x with an albumin content limited to 3 g/l).

Thus the similar and additive properties of albumin and its substitute make it possible to envisage a significant reduction in the albumin content and consequently the operating cost of such media.

Having regard to its properties and its low operating cost, the culture medium obtained from a composition according to the invention also opens up to a vast field of applications in the biotechnological industrial field, comprising in particular:

the culture of hybridomas producing monoclonal antibodies, in particular when these antibodies can be injected directly into humans. In this application, the medium according to the invention has the advantage of having no contaminating immunoglobulin.

the preparation of molecules produced by the culture of cells, in particular many lines, transfected or not with myelomas and CHO (Chinese Hamster Ovary cells), for therapeutic, vaccine, diagnostic or research purposes.

the preparation of biological entities by the culture of cells, in particular the production of viral vectors in the service of gene therapies.

All of the compositions or methods disclosed and claimed herein can be made and executed without undue experimentation in light of the present disclosure. While the composition and methods of this invention have been described in terms of specific embodiments, it will be apparent to those of skill in the art that variations may be applied to the compositions and methods and in the steps or in the sequence of steps of the methods described herein without departing from the concept, spirit and scope of the invention. All such similar substitutes and modifications apparent to those skilled in the art are deemed to be within the spirit, scope and concept of the invention.

What is claimed is:

1. A composition for the culture of cells or tissues comprising an albumin substitute, wherein the albumin substitute includes polyethylene glycol at a concentration of at least approximately 1% by weight.

2. The composition of claim 1, further comprising a transferrin substitute and an insulin substitute, where the composition is free from animal proteins other than recombinant proteins.

3. A composition according to claim 1, further comprising sodium erythorbate.

4. A composition comprising:
protein and serum free cell culture base; and
at least 1% polyethylene glycol by weight.

5. The composition of claim 4, wherein the composition is free from animal proteins other than recombinant proteins.

6. The composition of claim 4, wherein the polyethylene glycol substitutes for all or part of the amount of albumin normally required to culture cells or tissues in the composition.

7. The composition of claim 6, wherein the composition contains substantially no albumin.

8. The composition of claim 4, further comprising a transferrin substitute and an insulin substitute.

9. The composition of claim 4, further comprising sodium erythorbate.

10. The composition of claim 9, further comprising less than 0.1% by weight sodium erythorbate.

11. The composition of claim 10, further comprising less than 5*10⁻³% by weight sodium erythorbate.

12. The composition of claim 4, further comprising between 1% and 5% polyethylene glycol by weight.

13. The composition of claim 4, further comprising between 1% and 3% polyethylene glycol by weight.

14. The composition of claim 4, where the polyethylene glycol has a has a molecular weight of between 50 and 100,000 daltons.

15. The composition of claim 14, wherein the polyethylene glycol has a molecular weight of approximately 20,000 daltons.

16. The composition of claim 4, further comprising an additional substance complexed with the polyethylene glycol.

17. The composition of claim 16, wherein the additional substance is a liquid.

18. The composition of claim 16, wherein the additional substance is a growth factor.

19. The composition of claim 4, wherein the composition is in the form of a powder.

20. A cell or tissue culture medium comprising:
protein and serum free cell culture base; and
at least 10 g/l polyethylene glycol.

21. The medium of claim 21, wherein the medium is free from animal proteins other than recombinant proteins.

22. The medium of claim 21, wherein the polyethylene glycol substitutes for all or part of the amount of albumin normally required to culture cells or tissues in the medium.

23. The composition of claim 22, wherein the composition contains substantially no albumin.

24. The medium of claim 22, further comprising albumin.

25. The medium of claim 24, wherein the albumin concentration is less than 5 g/l.

26. The medium of claim 21, further comprising a transferrin substitute and an insulin substitute.

27. The medium of claim 21, further comprising sodium erythorbate.

28. The medium of claim 21, further comprising less than 1 g/l sodium erythorbate.

29. The medium of claim 28, further comprising less than 50 mg/l sodium erythorbate.

30. The medium of claim 21, further comprising between 10 g/l and 50 g/l polyethylene glycol.

31. The medium of claim 30, further comprising between 10 g/l and 30 g/l polyethylene glycol.

32. The medium of claim 21, where the polyethylene glycol has a molecular weight of between 50 and 100,000 daltons.

33. The medium of claim 32, wherein the polyethylene glycol has a molecular weight of approximately 20,000 daltons.

33. The medium of claim 21, further comprising an additional substance complexed with the polyethylene glycol.

34. The medium of claim 33, wherein the additional substance is a liquid.

35. The medium of claim 33, wherein the additional substance is a growth factor.

36. A method of culturing animal cells or tissues comprising:
preparing cell culture medium including:
a protein and serum free cell culture base; and
at least 10 g/l polyethylene glycol; and
placing animal cells or tissues to be cultured in the cell culture medium

37. The method of claim 36, wherein the cell culture medium further comprises sodium erythorbate.
